(19) United States
(12) Patent Application Publication
(10) Pub. No.: US 2021/0137443 A1
(43) Pub. Date: May 13, 2021

(54) SYSTEMS AND SURGICAL TECHNIQUES FOR MONITORING NERVE STATUS

(71) Applicant: JGMG BENGOCHEA, LLC, Jacksonville, FL (US)

(72) Inventor: Javier GARCIA-BENGOCHEA, Jacksonville, FL (US)

(21) Appl. No.: 16/610,628

(22) PCT Filed: May 4, 2018
(86) PCT No.: PCT/US2018/031210

§ 371 Date: Nov. 4, 2019

Related U.S. Application Data
(60) Provisional application No. 62/501,212, filed on May 4, 2017.

(51) Int. Cl. A61B 5/00

(52) U.S. Cl. CPC A61B 5/4041; A61B 5/407; A61B 2505/05; A61B 5/6847; A61B 5/746; A61B 5/4076

(57) ABSTRACT

A method for monitoring nerve tissue status during a surgical procedure using a monitoring system that includes a stimulating electrode and a sensing electrode each positioned in proximity to a nerve of a patient. Each electrode may be mono- or bi-polar. A first electrical stimulus is applied by the stimulating electrode and received by the sensing electrode. A monitoring unit determines a first transit time for the first stimulus. A second electrical stimulus is applied by the stimulating electrode and received by the sensing electrode. The monitoring unit determines a second transit time for the second stimulus. The monitoring unit then notifies a user if the second transit time is greater than the first transit time by a predetermined threshold, which may indicate degradation in nerve performance.

### FIELD

The present invention is directed to systems and surgical techniques for directly monitoring the status and condition of a nerve during a surgical procedure conducted proximate to the nerve. More particularly, the present application describes systems and techniques for direct nerve status monitoring during surgical procedures in the spine

### BACKGROUND

Various methods exist for monitoring nerves during surgical procedures. Such methods may determine when an electrified instrument is approaching a nerve, or the approximate location of a nerve. Specifically, according to such methods, a known voltage is communicated to a nerve stimulating instrument. As the instrument is manipulated within or on the patient, voltage passed through the instrument may evoke a detectible muscular response as detected by a specialized electromyogram (“EMG”) electrode when a nerve coupled to the responsive muscle is stimulated. The muscular response is recorded and an auditory and/or visual signal is produced which alerts the operator, such as a physician, that the instrument is considered to be near a nerve. At most, these devices provide information about the presence of a nerve, and in some cases its approximate vicinity relative to the stimulating instrument. However, these devices do not provide any direct information about the status of the nerve and any adverse effects or damage that may be inflicted on the nerve as a result of extended compression of the nerve caused by surgical instruments. A further drawback of these devices is that they are relied upon to stimulate a muscular response as the indirect means of confirming that a nerve is in the proximity of the surgical instruments. Thus, the patient's muscles cannot be numbed or temporarily paralyzed with, for example, neuromuscular blocking agents. Consequently, in order to utilize these indirect nerve monitoring instruments, it is an unavoidable reality that the surgical instruments, including the nerve stimulator, must be moved through tissue that is reactive, including perhaps the very muscle that is likely to be the responsive muscle. The passage of the instruments near or through a muscle that is active (i.e., not paralyzed) can result in disruptive contacting, twitching and resistance to the surgeon's efforts.

In view of the above described deficiencies, there is a need for surgical systems and techniques that do not rely on EMG electrodes placed in distant muscles, and instead enable direct measurement at or adjacent to the nerve of the presence of and status of a nerve that may be affected adversely during a surgical procedure. The need includes providing methods and instruments that allow direct monitoring in the presence of neuromuscular blockade agents to minimize the extent of muscle response to the stimulation.

### SUMMARY

In an embodiment, a method is provided for monitoring nerve tissue status during a surgical procedure using a monitoring system. In some embodiments, the monitoring system includes a surgical device having at least one of each of a stimulating electrode and a sensing electrode integral thereto. And one or both of the electrodes may be monopolar, bipolar or multi-polar. The surgical device is configured such that the stimulating electrode and the sensing electrode are electrically isolated from each other through the body of the surgical device. The stimulating electrode and sensing electrode of the surgical device are electrically connected to and in communication with a monitoring unit having a processor, a memory, and instructions stored within the memory.

The method includes positioning, by an operator, the stimulating electrode and sensing electrode in proximity to a nerve of a patient. The method further includes applying, by the stimulating electrode, a first electrical stimulus at a first time and receiving, by the sensing electrode, the first electrical stimulus at a second time. The method further includes determining, by the monitoring unit, a first transit time, based on the first time and the second time. The method further includes applying, by the stimulating electrode, a second electrical stimulus at a third time to the nerve and receiving, by the sensing electrode, the second electrical stimulus at a fourth time. The method further includes determining, by the monitoring unit, a second transit time, based on the third time and the fourth time and determining, by the monitoring unit, a difference between the first transit time and the second transit time. The method further includes notifying, by the monitoring unit, an operator if the second transit time is greater than the first transit time by more than a predetermined threshold.

In another embodiment, a method is provided for monitoring nerve tissue status during a surgical procedure using a monitoring system, the monitoring system including a first surgical device having at least one of a stimulating electrode and second surgical device having at least one of a sensing electrode. And one or both of the electrodes may be monopolar, bipolar or multi-polar. The stimulating electrode and sensing electrode being electrically connected to and in communication with a monitoring unit having a processor, a memory, and instructions stored within the memory.

The method includes positioning, by an operator, the stimulating electrode and sensing electrode in a spaced apart configuration in proximity to a nerve of a patient. The method further includes applying, by the stimulating electrode, a first electrical stimulus at a first time. The method further includes receiving, by the sensing electrode, the first electrical stimulus at a second time and determining, by the monitoring unit, a first transit time, based on the first time and the second time. The method further includes applying, by the stimulating electrode, a second electrical stimulus at a third time to the nerve and receiving, by the sensing electrode, the second electrical stimulus at a fourth time. The method further includes determining, by the monitoring unit, a second transit time, based on the third time and the fourth time and determining, by the monitoring unit, a difference between the first transit time and the second transit time.

In an embodiment, provided is a nerve tissue status monitoring system including a surgical probe (also referred to herein as a nerve status probe) having at least one of a stimulating electrode contact and a sensing electrode contact, where in some embodiments the stimulating electrode contact is connected via a non-electrically conductive bridge element to a sensing electrode contact. The probe may be integrated with or adapted to be integrated with a surgical device, and may be embodied as two separate probes comprising, respectively, the stimulating and the sensing electrodes. And one or both of the electrodes may be monopolar, bipolar or multi-polar. A stimulation and sensing module that is in communication with each of the electrode contacts and includes an electrical power source for producing and communicating an electrical current to the stimulating electrode contact, and a monitoring unit for receiving and processing signals from the sensing electrode contact, the monitoring unit having a processor, a memory, and instructions stored within the memory. When in use, the system, when in contact with a nerve at a surgical site via the probe, provides information about nerve status by measuring any change in a nerve conduction signal for the nerve that is based on the speed of transmission of current along the nerve between the stimulating and the sensing electrode contacts. A detected nerve conduction signal that is not diminished relative to an initial nerve conduction signal indicates that the nerve status is not impaired, and, a detected nerve conduction signal that is diminished relative to the initial nerve conduction signal indicates that the nerve status is impaired.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

Features and advantages of the general inventive concepts will become apparent from the following description made with reference to the accompanying drawings, including drawings represented herein in the attached set of figures, of which the following is a brief description.

FIG. 1 is a schematic depicting front, side and back views, from left to right, of an embodiment of a probe in accordance with the disclosure, the probe adapted in particular for a full open surgical procedure;

FIG. 2 is a schematic depicting front, side and back views, from left to right, of an embodiment of a monopolar probe in accordance with the disclosure, the probe adapted in particular for a full open surgical procedure;

FIG. 3 is a schematic depicting an alternative embodiment of a probe according to the disclosure, the probe engaged with a minimally invasive tubular retractor, the schematic showing from left to right: a representative outer surface of a minimally invasive tubular retractor; a first cross sectional view of the tubular retractor showing the probe engaged with the tubular retractor and in a front view of the probe, a second cross sectional view of the tubular retractor showing the probe engaged with the tubular retractor and in a orthogonal view of the probe showing the engagement means joining the probe to the tubular retractor; and an alternate view of the tubular retractor showing the probe engaged with the tubular retractor and in a back view of the probe showing the engagement means joining the probe to the tubular retractor;

FIG. 4 is a block diagram of a method of monitoring nerve status, according to an embodiment; and,

FIG. 5 is a block diagram of a method of monitoring nerve status, according to an embodiment.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning. Wherever possible, the same reference numbers will be used throughout the drawings to represent the same parts.

### DETAILED DESCRIPTION

The general inventive concepts will now be described with occasional reference to the exemplary embodiments of the invention. The general inventive concepts may be embodied in different forms and should not be construed as limited to the embodiments set forth herein. Rather, these embodiments are provided so that this disclosure will be thorough and complete, and will fully convey the scope of the general inventive concepts to those skilled in the art. Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts.

Nerve Status Probe

As described herein above, there is a need for devices and systems that overcome the shortcomings in the art pertaining to nerve monitoring. In particular, systems and techniques are needed to allow for direct monitoring of nerve status during a surgical procedure, whereby real time information about nerve status is provided to enable real time adjustments to the placement of or pressure applied by instruments and/or removal or adjustment of placement of implant, can be accomplished to minimize or eliminate nerve compression that would lead to long term damage and impairment to the patient.

Probes and Monitoring Systems

In accordance with the various embodiments, the system and methods described herein include use of a probe as a part of an overall monitoring system to provide real time direct assessment of the status of a nerve that is affected in a surgical field. The system is particularly useful in the context of a surgery in which the patient is partially or fully paralyzed using nerve blocking agents for all or a portion of the surgery. In particular, the system is useful to provide information about a nerve without reliance on an EMG electrode.

Referring now to the drawings, FIG. 1 shows a schematic depicting front, side and back views, from left to right, of an embodiment of a nerve status probe 100 in accordance with the disclosure. In the example of FIG. 1, the nerve status probe 100 includes a probe body 110 with a proximal manipulation end 120 and a distal tissue contacting end 130. The distal tissue contacting end 130 includes at least two spaced apart electrode contacts, including at least one stimulating electrode contact 140 and at least one sensing electrode contact 150. In some embodiments, the nerve status probe 100 is configured such that each of the at least to spaced apart electrode contacts are monopolar or multi-polar. In some particular embodiments, the probe is configured with two spaced apart electrode contacts, including at least one stimulating electrode contact 140 and at least one sensing electrode contact 150 wherein each of the electrode contacts is biopolar. In some other particular embodiments, the probe is configured with at least two spaced apart electrode contacts, including at least one stimulating electrode contact 140 and at least one sensing electrode contact 150 wherein at least one or more of each of the electrode contacts is biopolar. In yet other embodiments, the nerve status probe 100 is configured to operate in a monopolar, bipolar, or multi-polar configuration with respect to at least one or more of the at least two electrode contacts.

The electrode contacts 140, 150 may be integrated within the distal tissue contacting end 130, attached to or wound around or otherwise affixed to opposing portions of the probe body 110, or may be separate from the nerve status probe 100 but fixedly or releasably contacted with opposing portions of the probe body 110. In some embodiments, the opposing stimulating and sensing electrode contacts 140, 150 are each contacted with a non-electrically conductive bridge between them so that any stimulation of the nerve at the at least one stimulating electrode contact 140 is detectable only by the sensing electrode contact 150 as a result of transmission of the electrical impulse (e.g., voltage or current) along the nerve. In an alternate embodiment, there may be two probes 100, each of which includes at a distal tissue contacting end 130 one of the two electrode contacts 140, 150. An example of a nerve status probe 100 of the alternate embodiment is shown in FIG. 2. One or more electrical leads 160 are electrically connected to the electrode contacts 140, 150 and may extend internally and/or externally to the probe body 110. The leads 160 may be configured to electrically communicate with a remote monitoring unit (not shown).

An optional non-electrically conductive bridge element (not shown) that is positionable between and contactable with each of the two probes 100 may be used to maintain their relative positions at a selected distance apart.

FIG. 3 shows a schematic depicting an alternative embodiment of a surgical device 200 according to the disclosure, the surgical device 200 includes a nerve status probe 100 engaged with a minimally invasive tubular retractor 210, the schematic showing from left to right: a representative outer surface of the minimally invasive tubular retractor 210; a first cross sectional view of the tubular retractor 210 showing the nerve status probe 100 engaged with the tubular retractor 210 and in a front view of the surgical device 200, a second cross sectional view of the tubular retractor 210 showing the nerve status probe 100 engaged with the tubular retractor 210 and in a orthogonal view of the nerve status probe 100 showing the engagement means joining the nerve status probe 100 to the tubular retractor 210; and an alternate view of the tubular retractor 210 showing the nerve status probe 100 engaged with the tubular retractor 210 and in a back view of the nerve status probe 100 showing the engagement means joining the nerve status probe 100 to the tubular retractor 210 The monitoring system includes two electrode contacts that are positionable relative to one another at a fixed distance apart and placed in proximity to a nerve. In some embodiments, one or more of the electrode contacts are in electrical communication with the nerve. In other embodiments, one or more of the electrode contacts may be in contact with the nerve.

In use, application of an electrical stimulus (e.g. voltage pulse or current pulse) to one of the stimulating electrode contacts 140 will enable transmission of the stimulus along a contacted nerve. The other electrode contact is a sensing electrode 150 that can detect and transmit information about the nerve impulse. The two electrode contacts 140, 150 may be connected via a non-electrically conductive bridge element, or they may be placed independently in the tissue. In either embodiment, the distance between the electrode contacts is known so that information about the transmitted impulse can be calculated, including any change in the impulse between an initial stimulation and a subsequent stimulation which can be reflected in at least a change in the transmission time between the electrode contacts 140, 150 or a change in the intensity of the impulse or both at the sensing electrode contact 150. The system also includes a stimulation and sensing module that is in communication with each of the electrode contacts and includes an electrical power source for producing and communicating an electrical current to the stimulating electrode contact, and sensing circuitry for receiving and processing signals from the sensing electrode contact. In some embodiments, the stimulation and sensing unit may be integral with the monitoring unit. In the various embodiments, stimulation is achieved based on values that are customary in the nerve status monitoring art, which range from >0 to 100 mA, with increments of 0.1 mA up to 20 mA and 1 mA increments thereafter. Values for voltage vary based on impedance due to tissue, electrode and contacts which can average about 1 kohm, for voltages typically in the range from about 0.1 V to about 100 V. In some embodiments, the electrical stimulus voltage may be about 0.1 V to about 2 V. In some embodiments, the electrical stimulus current may be about 0.1 mA to about 5 mA. In some embodiments, the duration of the electrical stimulus may be about 0.05 milliseconds to about 3 milliseconds. In one embodiment, the duration of the electrical stimulus may be about 0.1 milliseconds to about 0.3 milliseconds.

When the nerve status probe 100 is in electrical communication with a nerve at a surgical site, the system provides information about nerve status by measuring any change in a nerve conduction signal that is based on at least one or more of the speed of transmission of the impulse along the nerve between the two electrode contacts 140, 150 and the intensity of the impulse. In some embodiments, paralysis may be induced in the patient during surgery. In some embodiments, the electrical stimulus detected by the sensing electrode does not result in muscular stimulation and/or response, in contrast to electromyography measurements. In some embodiments, the sensing electrode may be positioned remotely along the nerve from the surgical site. For example, the stimulating electrode may be in proximity to a nerve and surgical instruments applying pressure to surrounding tissue. A sensing electrode may be positioned along the same nerve remotely in the patient, such as along a leg.

In some examples, when a detected nerve conduction signal is not diminished relative to an initial nerve conduction signal, the reading indicates that the nerve status is not impaired. And in some examples, when a detected nerve conduction signal is diminished relative to the initial nerve conduction signal the reading indicates that the nerve status is impaired. If the signal has diminished by more than a predetermined threshold, the monitoring unit may notify an operator. The operator may then make one or more adjustments to the position of the surgical instruments and implants to alleviate damaging pressure or other contact on the nerve.

In some embodiments, the repositioning of the surgical instruments may include repositioning one or more instruments having a stimulating and/or sensing electrode. In such situations an updated baseline nerve conduction measurement may need to be obtained. Subsequent measurements using the repositioned electrode may then be compared to the updated baseline measurement. In other embodiments, the surgical instruments applying undesirable pressure on a nerve, and thus relocated, may be different from the surgical instruments having a stimulating and/or sensing electrode.

The system may also include data capture, data processing, and other functionalities as may be embodied in a computer or other device. And the system may further include a program or algorithm that automatically administers an electrical stimulation at predetermined fixed or variable intervals. The system may further include any of a variety of feedback signals that may include predetermined thresholds for detected changes in the transmitted impulse. In some examples, the system may automatically administer impulses at fixed minute intervals during a surgical procedure, and may provide feedback to an operator if there is a loss of a sensed impulse, or if there is an increase in transmission time that is greater than a predetermined threshold or if there is an increase or a decrease in the intensity of the impulse below or above predetermined thresholds.

It will be appreciated that embodiments of instruments useful according to the methods described herein are not limited to generic probes, or dilators or retractors as shown in the drawings, and may instead be implemented with devices and equipment such as, for example, implants such as pedicle screws, intra disc devices, and other spinal implants, as well as pedicle probes, cutting devices, rasps, trocars, spreaders, distracters, shims, scrapers, chisels, disc cutters, curettes, suction probes, tamps, and the like, which are inserted into innervated areas of the body that where the persistent presence of the implant or instrument could give rise to nerve damage if contact or compression is not relieved. For example, electrode contacts could be located at opposing positions along an edge of such devices. In other embodiments, two or more devices that include at least one of a stimulating and at least one of a sensing electrode contact may be placed at a distance apart such that the direct monitoring of a contacted nerve according to the methods described herein can be achieved.

The system also includes at least one electrode lead 160 that passes from the proximal to the distal end of the probe body 110, the electrode lead 160 is in communication at the proximal end with an electrical stimulation delivery component and at the distal end with a stimulating electrode contact 140. The system also includes at least one electrical sensor that passes from the proximal to the distal end of the body, the electrical sensor in communication at the proximal end of the elongate body with a sensing transmission component and at the distal end with a sensing electrode contact 150. The system also includes an interface for communication between, respectively, each of the stimulating and the sensing transmission components, and a monitoring unit. The system also includes a stimulation and sensing module that includes an electrical power source for producing and communicating an electrical current through the electrical stimulation delivery component and along the electrode to the stimulating electrode contact 140, and sensing circuitry for receiving and processing signals through the sensing transmission component from the sensing electrode contact 150.

Embodiments of the systems may be implemented in code and may be stored on a storage medium having stored thereon instructions which can be used to program a system to perform the instructions. The storage medium may be non-transitory and may include, but is not limited to, any type of disk including floppy disks, optical disks, solid state drives (SSDs), compact disk read-only memories (CD-ROMs), compact disk rewritables (CD-RWs), and magneto-optical disks, semiconductor devices such as read-only memories (ROMs), random access memories (RAMs) such as DRAMs, static random access memories (SRAMs), erasable programmable read-only memories (EPROMs), flash memories, electrically erasable programmable read-only memories (EEPROMs), magnetic or optical cards, or any other type of media suitable for storing electronic instructions. Such computer-readable or computer-usable storage medium or media is (are) considered to be part of an article (or article of manufacture). An article or article of manufacture can refer to any manufactured single component or multiple components.

Embodiments of the invention may be described herein with reference to data such as instructions, functions, procedures, data structures, application programs, configuration settings, code/s, and the like. When the data is accessed by a machine, the machine may respond by performing tasks, defining abstract data types, establishing low-level hardware contexts, and/or performing other operations, as described in greater detail herein. The data may be stored in volatile and/or non-volatile data storage. The terms “code” or “program” cover a broad range of components and constructs, including applications, drivers, processes, routines, methods, modules, and subprograms. Thus, the terms “code” or “program” may be used to refer to any collection of instructions which, when executed by a processing system, performs a desired operation or operations. In addition, alternative embodiments may include processes (in code or otherwise) that use fewer than all of the disclosed operations, processes that use additional operations, processes that use the same operations in a different sequence, and processes in which the individual operations disclosed herein are combined, subdivided, or otherwise altered.

Methods

In accordance with some embodiments, a surgical technique for monitoring nerve tissue includes inserting a probe, such as an embodiment of a surgical retractor shown in the drawings, into a surgical site and adjacent soft tissue that contains a nerve (e.g., psoas muscle during a fusion procedure).

In use, one or more probes 100 are directed into the surgical site, with the distal tissue contacting end 130 placed in proximity to a surgical target within the surgical site and in direct contact with soft tissue adjacent to the surgical target. An electrical stimulus is communicated to the at least one stimulating electrode contact 140 and any response to the at least one sensing electrode contact 150 is detected, wherein if a response is detected, the probe is confirmed adjacent to and in electrical communication with a nerve.

The locus of a nerve may initially be determined either with the probe/s or with a separate neuromonitoring device at the time of or prior to placement of the probe/s. An initial (baseline) nerve conduction signal or impulse is captured and calculated (time or intensity of transmission, or such other non-limiting properties as may be determined) involving quantifying the intensity of the signal transmission between the stimulating electrode contact 140 and the sensing electrode contact 150. Thereafter, the step of communicating an electrical stimulus to the at least one stimulating electrode contact 140 and monitoring for a response on the at least one sensing electrode contact 150, is repeated one or more times, wherein a detected nerve conduction signal that is not diminished relative to the initial nerve conduction signal indicates that the nerve status is not impaired, and, wherein a detected nerve conduction signal that is diminished relative to the initial nerve conduction signal indicates that the nerve status is impaired.

In some examples according to the method, when a diminished nerve conduction signal is detected, the method includes the further steps of removing or adjusting the position of surgical implants or instruments, and repeating the prior stimulating and detecting steps to assess the status of the nerve for any changes that would indicate persistent compromise, improved status, or loss of contact with the nerve.

In some examples according to the method, when there is no diminished nerve conduction signal detected, the method comprises the further steps of maintaining the position of surgical implants or instruments, and repeating the prior stimulating and detecting steps to assess the status of the nerve for any changes that would indicate persistent compromise, improved status, or loss of electrical communication with the nerve.

In some examples according to the method, the surgical procedure is selected from an open and a minimally invasive spinal surgery.

In some examples according to the method, the probe is one of affixed to and integrated with a surgical retractor. In some specific examples according to the method, the retractor has an elongate body and the distal tissue contacting end has a flared substantially planer blade defined by two lateral edges, each lateral edge comprising one of the electrode contacts, the blade forming the non-electrically conductive bridge element. In some embodiments, the blade has a distal edge that is one of flat and curved. In other embodiments, a probe may be one or a combination of medical devices selected from probes, distractors, screws, retractors, shims and the like.

It will be appreciated that in some surgeries, more than one nerve may be affected by the procedure, and thus, more than one probe and system (e.g., one system per nerve), may be used to monitor the status of each nerve during the course of the procedure.

FIG. 4 is a block diagram of a method of monitoring nerve status 300. In block 310, positioning, by an operator, the stimulating electrode and sensing electrode in proximity to a nerve of a patient. At block 320, applying, by the stimulating electrode, a first electrical stimulus at a first time. At block 330, receiving, by the sensing electrode, the first electrical stimulus at a second time. At block 340, determining, by the monitoring unit, a first transit time, based on the first time and the second time. At block 350, applying, by the stimulating electrode, a second electrical stimulus at a third time to the nerve. At block 360, receiving, by the sensing electrode, the second electrical stimulus at a fourth time. At block 370, determining, by the monitoring unit, a second transit time, based on the third time and the fourth time. At block 380, determining, by the monitoring unit, a difference between the first transit time and the second transit time. At block 390, notifying, by the monitoring unit, an operator if the second transit time is greater than the first transit time by more than a predetermined threshold.

FIG. 5 is a block diagram of a method of monitoring nerve status 400. In block 410, positioning, by an operator, the stimulating electrode and sensing electrode in a spaced apart configuration in proximity to a nerve of a patient. At block 420, applying, by the stimulating electrode, a first electrical stimulus at a first time. At block 430, receiving, by the sensing electrode, the first electrical stimulus at a second time. At block 440, determining, by the monitoring unit, a first transit time, based on the first time and the second time. At block 450, applying, by the stimulating electrode, a second electrical stimulus at a third time to the nerve. At block 460, receiving, by the sensing electrode, the second electrical stimulus at a fourth time. At block 470, determining, by the monitoring unit, a second transit time, based on the third time and the fourth time. At block 480, determining, by the monitoring unit, a difference between the first transit time and the second transit time.

In accordance with some embodiments, a nerve tissue status monitoring system that is used in the methods according to the disclosure includes a surgical device having at least one stimulating electrode contact that is connected via a non-electrically conductive bridge element to a sensing electrode contact, and a stimulation and sensing module that is in communication with each of the electrode contacts. In some embodiments the one or more of each of the stimulating and sensing electrode contacts are on the same surgical instrument and in some embodiments they are on different instruments. In some instances, more than one of each at least one of the stimulating electrode contacts is on a particular surgical instrument. And according to some embodiments where at least one stimulating electrode contact is on a first instrument and at least one sensing electrode contact is on a second instrument, it will be understood that the instruments may be in contact with the same tissue in a surgical field, or with different tissue in the field and in some embodiments, the first instrument is in the surgical field and the second instrument is downstream along the path of at least one nerve that is in the surgical field and contacted directly or indirectly by the first instrument. In some embodiments, the downstream instrument includes an electrode comprising an EMG electrode. According to the various uses, when the device is in electrical contact with a nerve at a surgical site, it provides information about nerve status by measuring any change in a nerve conduction signal, wherein a detected nerve conduction signal that is not diminished relative to an initial nerve conduction signal indicates that the nerve status is not impaired, and, wherein a detected nerve conduction signal that is diminished relative to the initial nerve conduction signal indicates that the nerve status is impaired. In various embodiments, the stimulating electrode and sensing electrode of the surgical device are electrically connected to and in communication with an electrical power source for producing and communicating an electrical current to the stimulating electrode contact, and a monitoring unit for receiving and processing signals from the sensing electrode contact, the monitoring unit having a processor, a memory, and instructions stored within the memory. In some embodiments, one or both of the stimulating and sensing electrodes is bipolar.

In some examples, the system may be used in the context of monitoring the effects on a nerve of a surgical instrument that is deployed in the surgical field, such as a clamp, retractor, distractor, or other manipulating instrument, which instrument may be retained in place during all or a portion of the surgery wherein its position may need to be adjusted from time to time to avoid extended and damaging compression or other impact on a nerve. In one example, such a procedure may include retroperitoneal access to the spine wherein a retractor or other instrument is used to displace tissue, such as the psoas muscle, away from a vertebra in the lumber spine in a manner that could compress a nerve branch of the lumbar plexus.

In other examples, the system may be used in the context of verifying the placement of an implant that is intended to be left in the patient after the procedure, wherein refinements in the positioning of the implant prior to closing the surgical field will help to minimize impingement of the implant on the nerve and thereby minimize long term adverse effects including pain, and nerve damage. In one example, such a procedure may include placement of a pedicle screw in the pedicle of a vertebra.

While in some embodiments, the systems and techniques are particularly useful for spinal procedures involving lateral access, the embodiments of the present invention are not limited to use in a posterior-lateral approach for spinal surgery, and may also be used in many other surgical approaches, including approaches to the spine, such as anterior (ALIF), posterior (PLIF), transverse (TLIF), and extreme lateral (XLIF). Embodiments of the present invention should also not be limited to the spine and may be used in other orientations and other surgical sites within the body where monitoring of nerve status and protection of one or more nerves from compression and other surgical damage is desirable. Some particular examples include prostate and other urogenital surgeries, and extremities surgeries, such as of the shoulder and the knees.

As used herein, the singular forms “a,” “an,” and “the” are intended to include the plural forms as well, unless the context clearly indicates otherwise. The term “proximal” as used in connection with any object refers to the portion of the object that is closest to the operator of the object (or some other stated reference point), and the term “distal” refers to the portion of the object that is farthest from the operator of the object (or some other stated reference point). The term “operator” means and refers to any professional or paraprofessional who delivers clinical care to a medical patient, particularly in connection with the delivery of care.

With respect to any references herein that may be made relative to a human patient, the terms “cephalad,” “cranial” and “superior” indicate a direction toward the head, and the terms “caudad” and “inferior” indicate a direction toward the feet. Likewise, the terms “dorsal” and “posterior” indicate a direction toward the back, and the terms “ventral” and “anterior” indicate a direction toward the front. And the term “lateral” indicates a direction toward a side of the patient, the term “medial” indicates a direction toward the mid line of the patient, and away from the side, the term “ipsalateral” indicates a direction toward a side that is proximal to the operator or the object being referenced, and the term “contralateral” indicates a direction toward a side that is distal to the operator or the object being referenced. More generally, all terms providing spatial references to anatomical features shall have meaning that is customary in the art.

Unless otherwise indicated, all numbers expressing quantities, properties, and so forth as used in the specification, drawings and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

References to visualization using radiography as described in the exemplary techniques herein are merely representative of the options for the operator to visualize the surgical field and the patient in one of many available modalities. It will be understood by one of ordinary skill in the art that alternate devices and alternate modalities of visualization may be employed depending on the availability in the operating room, the preferences of the operator and other factors relating to exposure limits. While confirmation of instrument placement in the course of the technique is appropriate, the frequency and timing relative to the sequence of steps in the technique may be varied and the description herein is not intended to be limiting. Accordingly, more or fewer images, from more or fewer perspectives, may be collected.

One of ordinary skill will appreciate that references to positions in the body are merely representative for a particular surgical approach, and according to the exemplary embodiments herein, are suitable for any number of animal patients, including humans and other species. Of course, the type of surgery, target tissue, and species of patient may be different than is disclosed in the exemplary embodiments described herein. Further, all references herein are made in the context of the representative images shown in the drawings. Fewer or additional generic instruments may be used according to the preference of the operator. Moreover, references herein to specific instruments are not intended to be limiting in terms of the options for use of other instruments where generic options are available, or according to the preference of the operator.

Thus, while the disclosed embodiments have been described and depicted in the drawings in the context of the human spine, it should be understood by one of ordinary skill that all or various aspects of the embodiments hereof may be used in connection with other species and within any species on other parts of the body where deep access within the tissue is desirable.

Further, while various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed.

Those skilled in the art may readily adopt one or more of the inventive aspects, concepts and features into additional embodiments and uses within the scope of the general inventive concepts, even if such embodiments are not expressly disclosed herein. Additionally, even though some features, concepts and aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated.

Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A method for monitoring nerve tissue status during a surgical procedure using a monitoring system, the monitoring system including a surgical device having a stimulating electrode and a sensing electrode integral thereto, the surgical device configured such that the stimulating electrode and the sensing electrode are electrically isolated from each other through the body of the surgical device, the method comprising:
positioning, by an operator, the stimulating electrode and sensing electrode in proximity to a nerve of a patient;
applying, by the stimulating electrode, a first electrical stimulus at a first time;
receiving, by the sensing electrode, the first electrical stimulus at a second time;
determining, by the monitoring unit, a first transit time, based on the first time and the second time;
applying, by the stimulating electrode, a second electrical stimulus at a third time to the nerve;
receiving, by the sensing electrode, the second electrical stimulus at a fourth time;
determining, by the monitoring unit, a second transit time, based on the third time and the fourth time;
determining, by the monitoring unit, a difference between the first transit time and the second transit time;
notifying, by the monitoring unit, an operator if the second transit time is greater than the first transit time by more than a predetermined threshold.

2. The method of claim 1, the stimulating electrode and sensing electrode of the surgical device being electrically connected to and in communication with an electrical power source for producing and communicating an electrical current to the stimulating electrode contact, and a monitoring unit for receiving and processing signals from the sensing electrode contact, the monitoring unit having a processor, a memory, and instructions stored within the memory.

3. The method of claim 1, wherein one or both of the stimulating and sensing electrodes is bipolar.

4. The method of claim 1, wherein the patient is under general or localized paralysis at a location in proximity to the stimulating electrode.

5. The method of claim 1, wherein the stimulating electrode and sensing electrode are positioned in proximity to a surgical target within a surgical site and in direct contact with soft tissue adjacent to the surgical target.

6. The method of claim 5, further comprising removing or adjusting the position of one or more surgical implants or devices, based on the difference between the first transit time and the second transit time.

7. The method of claim 1, wherein the surgical device includes a surgical retractor.

8. The method of claim 7, wherein the surgical retractor includes an elongate body having a distal tissue contacting end including a flared substantially planer blade defined by two lateral edges, each lateral edge comprising one of the two sensors, the blade forming the non-electrically conductive bridge element.

9. The method of claim 8, wherein the blade has a distal edge that is one of flat and curved.

10. A method for monitoring nerve tissue status during a surgical procedure using a monitoring system, the monitoring system including a first surgical device having a stimulating electrode and second surgical device having a sensing electrode, the method comprising:
positioning, by an operator, the stimulating electrode and sensing electrode in a spaced apart configuration in proximity to a nerve of a patient;
applying, by the stimulating electrode, a first electrical stimulus at a first time;
receiving, by the sensing electrode, the first electrical stimulus at a second time;
determining, by the monitoring unit, a first transit time, based on the first time and the second time;
applying, by the stimulating electrode, a second electrical stimulus at a third time to the nerve;
receiving, by the sensing electrode, the second electrical stimulus at a fourth time;
determining, by the monitoring unit, a second transit time, based on the third time and the fourth time;
determining, by the monitoring unit, a difference between the first transit time and the second transit time.

11. The method of claim 10, the stimulating electrode and sensing electrode of the surgical device being electrically connected to and in communication with an electrical power source for producing and communicating an electrical current to the stimulating electrode contact, and a monitoring unit for receiving and processing signals from the sensing electrode contact, the monitoring unit having a processor, a memory, and instructions stored within the memory.

12. The method of claim 11, further comprising notifying, by the monitoring unit, an operator if the second transit time is greater than the first transit time by more than a predetermined threshold.

13. The method of claim 11 wherein none of the electrodes is an EMG electrode.

14. The method of claim 12, further comprising removing or adjusting the position of one or more surgical implants or devices, based on the difference between the first transit time and the second transit time.

15. The method of claim 13, further comprising:
determining a new baseline transit time by, applying a new first electrical stimulus at a new first time; receiving, by the sensing electrode, the first electrical stimulus at a new second time;
determining, by the monitoring unit, a new first transit time, based on the new first time and the new second time.

16. The method of claim 10, wherein one or both of the stimulating and sensing electrodes is bipolar.

17. The method of claim 11, wherein the patient is under general or localized paralysis at a location in proximity to the stimulating electrode.

18. The method of claim 11, wherein the stimulating electrode and sensing electrode are positioned in proximity to a surgical target within a surgical site and in direct contact with soft tissue adjacent to the surgical target.

19. The method of claim 18, wherein the stimulating electrode is positioned in proximity to a surgical target within a surgical site that is in the spine and in direct contact with soft tissue adjacent the surgical target, and wherein the sensing electrode is positioned in proximity to soft tissue that is downstream from the stimulating electrode and adjacent one of a muscle innervated by a nerve in the soft tissue adjacent the surgical site, and a nerve in the soft tissue adjacent the surgical site.

20. The method of claim 11, wherein the first surgical device includes a surgical retractor.

21. The method of claim 11, wherein the second surgical device includes a surgical probe.

22. The method of claim 11, wherein the surgical procedure includes minimally invasive spinal surgery.

23. The method of claim 11, wherein the stimulating electrode is integrated with the first surgical device.

24. The method of claim 23, wherein the first surgical device includes a surgical retractor.

25. The method of claim 24, wherein the surgical retractor includes an elongate body having a distal tissue contacting end including a flared substantially planer blade defined by two lateral edges, each lateral edge comprising one of the electrodes, the blade forming the non-electrically conductive bridge element.

26. The method of claim 25, wherein the blade has a distal edge that is one of flat and curved.

27. The method of claim 11, wherein the electrical stimulus duration is between about 0.05 milliseconds and 0.3 milliseconds.

28. The method of claim 11, wherein the electrical stimulus is between 0.1 milliamperes and 5 milliamperes.
